(19) United States
(12) Patent Application Publication
(10) Pub. No.: US 2023/0122165 A1
(43) Pub. Date: Apr. 20, 2023

(54) Head and Facial Protective Device

(71) Applicant: April Surgical Products, LLC, Traverse City, MI (US)

(72) Inventors: Kristin Bauman, Traverse City, MI (US); David Gomez, Holly Springs, NC (US); Nathan Winder, Stilwell, KS (US); Luke Truax, Kailua, HI (US)

(21) Appl. No.: 17/969,433

(22) Filed: Oct. 19, 2022

Related U.S. Application Data
(60) Provisional application No. 63/257,443, filed on Oct. 19, 2021.

(51) Int. Cl. A61B 90/00; A61F 9/04

(52) U.S. Cl. CPC A61B 90/03; A61F 9/04; A61B 2090/036; A61B 2090/502

(57) ABSTRACT

Embodiments disclosed herein include devices and methods of using devices for protecting a face and/or head of an individual receiving medical treatment in a supine or reclined position. The device includes a body having a top portion with an exterior surface and a base portion with an interior surface. The top portion includes opposing side edges, the base portion includes opposing bottom side edges extending outwardly from the top side edges. The base portion is flexible and is configured to wrap partially around the individual’s head, while an aperture extends between the exterior surface and the interior surface to provide access to at least one of the individual’s eyes, nose, or mouth. A strap spans the side edges, wraps behind the base portion and the individual’s head, and detachably couples to the exterior surface along a plurality of connection positions along the side edges.

### CROSS REFERENCE TO RELATED APPLICATIONS

This application claims priority to and the benefit of U.S. Provisional Pat. Application No. 63/257,443 filed Oct. 19, 2021, the entire contents of which are incorporated by reference.

### BACKGROUND

Protecting a patient’s face during a medical treatment (e.g., a surgery) when the patient is lying in the supine or the reclined position is an important concern. As described herein, the supine position is when the patient is lying horizontally on their back and their face and torso are facing substantially upward. In addition, the reclined position includes positions where the patient is lying on their back such that the patient’s face is oriented within 45 degrees of horizontal. In either the supine or the reclined positions, localized or uneven pressures applied to a patient’s face during a medical treatment can lead to decreased blood flow, compression injuries, and to nerve damage. For example, pressures applied to the ocular structures can cause severe nerve damage and even blindness. Additionally, compression injuries may also occur for example to portions of the forehead, nose, ears, cheeks, and mouth. Once general anesthesia is administered, a patient becomes progressively less able to respond to stimulus that would otherwise trigger discomfort or pain. Therefore constant vigilance is needed by the anesthesia provider and by the other clinical staff to protect the patient’s vital facial and head structures during medical treatments.

U.S. Pat. No. 9,723,881 (the ‘881 Patent), the entirety of which is incorporated herein by reference, disclosed a protective device for protecting the face and/or head of a patient receiving medical treatment in the supine position. The ‘881 Patent discloses a device including a face portion having an exterior surface, an interior surface, and an aperture extending from the exterior surface to the interior surface. The device includes a pair of opposed flexible side portions extending outwardly from the face portion. The face portion was configured to be positioned about the patient’s face with at least a portion of the face exposed by the aperture. The side portions were configured to be wrapped around and completely surround the patient’s head such that at least one of the side portions was positioned underneath the patient’s head. While the ‘881 Patent has provided many valuable solutions to users treating patients in the supine position, the clinical usage has also shown that the user’s experience, particularly for example an anesthesia provider’s experience, can be improved. For example, the side portions that were configured to be wrapped completely around the patient’s head limited the utility of the device. Accordingly, there is a need for an improved head and facial protection device as described herein.

### SUMMARY

The present disclosure relates generally to a protective device for a patient’s head and/or face that may be used during surgery and other procedures.

In light of the present disclosure, and without limiting the scope of the disclosure in any way, in a first aspect of the present disclosure, which may be combined with any other aspect listed herein unless specified otherwise, a protective device for protecting a face and/or head of an individual receiving medical treatment in a supine or reclined position. The protective device includes a body including a top portion with an exterior surface and a base portion with an interior surface. The top portion includes opposing side edges, and the base portion includes opposing bottom side edges extending outwardly from the top side edges. The base portion is flexible and is configured to wrap partially around the individual’s head, and the body includes an aperture extending between the exterior surface and the interior surface to provide access to at least one of the individual’s eyes, nose, or mouth. A strap spanning the side edges of the top portion of the body and configured to wrap behind the base portion of the body and the individual’s head to secure the interior surface of the base portion into proximate contact with the face and/or head, and wherein the strap is configured to detachably couple to the exterior surface of the top portion along a plurality of connection positions.

In accordance with a second aspect of the present disclosure, which may be used in combination with any other aspect listed herein unless stated otherwise, the protective device further includes an eye protection shield configured to detachably couple to the exterior surface of the top portion and to at least partially obstruct the aperture.

In accordance with a third aspect of the present disclosure, which may be used in combination with any other aspect listed herein unless stated otherwise, the plurality of connection positions includes four connection points on the exterior surface of the top portion, and wherein the strap is formed from a continuous piece of material.

In accordance with a forth aspect of the present disclosure, which may be used in combination with any other aspect listed herein unless stated otherwise, the plurality of connection positions includes four segments of hook fabric on the exterior surface of the top portion, and the strap is formed from a continuous piece of loop fabric and is configured to equalize pressure between the plurality of connection positions.

In accordance with a fifth aspect of the present disclosure, which may be used in combination with any other aspect listed herein unless stated otherwise, the top portion is monolithically formed with the base portion.

In accordance with a sixth aspect of the present disclosure, which may be used in combination with any other aspect listed herein unless stated otherwise, the top portion is thicker than the base portion.

In accordance with a seventh aspect of the present disclosure, which may be used in combination with any other aspect listed herein unless stated otherwise, the exterior surface further includes a slit extending partially through a thickness of the top portion, wherein opposing surfaces forming the slit are configured to frictionally grip tubing or electrically conductive wires placed within the slit.

In accordance with a eighth aspect of the present disclosure, which may be used in combination with any other aspect listed herein unless stated otherwise, the top portion and the base portion are adhesively coupled.

In accordance with a ninth aspect of the present disclosure, which may be used in combination with any other aspect listed herein unless stated otherwise, the top portion is formed from a first foam that is stiffer than a second foam forming the base portion.

In accordance with a tenth aspect of the present disclosure, which may be used in combination with any other aspect listed herein unless stated otherwise, a protective device for protecting a face and/or head of an individual receiving medical treatment in a supine or reclined position. The protective device including a body comprising a top portion with an exterior surface and a base portion with an interior surface. The top portion includes opposing side edges, and the base portion includes opposing bottom side edges extending outwardly from the top side edges. The base portion is flexible and is configured to wrap partially around the individual’s head. The top portion is not configured to wrap partially around the individual’s head. The body includes a first aperture extending between the exterior surface and the interior surface to provide access to the individual’s eyes, and a second aperture extending between the exterior surface and the interior surface to provide access to the individual’s nose and mouth. The protective device also includes a strap spanning the side edges of the top portion of the body and configured to wrap behind the individual’s head to secure the interior surface of the base portion into proximate contact with the face and/or head. The strap is configured to detachably couple to the exterior surface of the top portion along a plurality of connection positions.

In accordance with an eleventh aspect of the present disclosure, which may be used in combination with any other aspect listed herein unless stated otherwise, the plurality of connection positions includes four segments of hook fabric on the exterior surface of the top portion, and the strap is formed from a continuous piece of loop fabric.

In accordance with a twelfth aspect of the present disclosure, which may be used in combination with any other aspect listed herein unless stated otherwise, the top portion is monolithically formed with the base portion.

In accordance with a thirteenth aspect of the present disclosure, which may be used in combination with any other aspect listed herein unless stated otherwise, the exterior surface further includes a slit extending partially through a thickness of the top portion, and opposing surfaces forming the slit are configured to frictionally grip tubing or electrically conductive wires placed within the slit.

In accordance with a fourteenth aspect of the present disclosure, which may be used in combination with any other aspect listed herein unless stated otherwise, the top portion and the base portion are adhesively coupled.

In accordance with a fifteenth aspect of the present disclosure, which may be used in combination with any other aspect listed herein unless stated otherwise, the top portion is formed from a first foam that is stiffer than a second foam forming the base portion.

In accordance with a sixteenth aspect of the present disclosure, which may be used in combination with any other aspect listed herein unless stated otherwise, further including an eye protection shield configured to detachably couple to the exterior surface of the top portion and fully obstruct the first aperture.

In accordance with a seventeenth aspect of the present disclosure, which may be used in combination with any other aspect listed herein unless stated otherwise, the eye protection shield is formed from a clear and fog-free material.

In accordance with an eighteenth aspect of the present disclosure, which may be used in combination with any other aspect listed herein unless stated otherwise, a method of protecting a face and/or head of an individual receiving medical treatment in a supine or reclined position. The method includes providing a device with a body including a top portion and a base portion. The top portion includes opposing side edges, and the base portion includes opposing bottom side edges extending outwardly from the top side edges. The method further includes providing a strap behind the individual’s head, and opposing ends of the strap extend beyond the sides of the individual’s head. The method further includes placing an interior surface of the base portion into proximate contact with the face and/or head of the individual, positioning the body so that an aperture provides access at least one of the individual’s eyes, nose, or mouth to an exterior surface, and wrapping at least a portion of the base portion partially around the individual’s head. The top portion is not configured to wrap partially around the individual’s head. Still further the method includes attaching the ends of the strap to the side edges of the top portion of the device and coupling the strap to the exterior surface of the top portion along a plurality of connection positions to secure the body to the individual’s face and/or head.

In accordance with a nineteenth aspect of the present disclosure, which may be used in combination with any other aspect listed herein unless stated otherwise, a method further including coupling an eye protection shield to the exterior surface of the top portion to at least partially obstruct the aperture.

In accordance with a twentieth aspect of the present disclosure, which may be used in combination with any other aspect listed herein unless stated otherwise, a method further including pressing tubing or electrically conductive wires into a slit formed in the exterior surface of the top portion, wherein the slit extends partially through a thickness of the top portion, and holding the tubing or electrically conductive wires in a fixed position with friction of opposing surfaces forming the slit.

Additional features and advantages of the disclosed apparatus, system and method are described in, and will be apparent from, the following detailed description and figures. The features and advantages described herein are not all-inclusive and, in particular, many additional features and advantages will be apparent to one of ordinary skill in the art in view of the figures and description. Also, any particular embodiment does not have to have all of the advantages listed herein. Moreover, it should be noted that the language used in the specification has been selected for readability and instructional purposes, and not to limit the scope of the inventive subject matter.

### BRIEF DESCRIPTION OF THE DRAWINGS

These and other features, aspects, and advantages of certain embodiments will become better understood when the following detailed description is read with reference to the accompanying drawings in which like characters represent like parts throughout the drawings, wherein:

FIG. 1 is a perspective view of a protective device for protecting a face and/or head of an individual receiving medical treatment in a supine or reclined position, according to one or more embodiments;

FIG. 2 is a top view of the protective device of FIG. 1 including an eye protection shield;

FIG. 3 is a perspective view of a body of the protective device of FIG. 2 in a relaxed state when not affixed to a patient;

FIG. 4 is another perspective view of the protective device of FIG. 3 shown without the eye protection shield;

FIG. 5 is a perspective view of the eye protection shield;

FIG. 6 is a perspective view of a strap, according to one or more embodiments;

FIG. 7 is a perspective view of the strap of FIG. 6 affixing an anesthesia face mask to a patient; and

FIG. 8 is a perspective view of a further embodiment of the protective device of FIG. 1.

### DETAILED DESCRIPTION

The present disclosure relates generally to a protective device for a patient’s head and/or face that may be used during surgery and other procedures. Specifically, the present disclosure provides protective devices and methods for protecting the head and/or face of a patient undergoing a procedure while lying in the supine position. In addition, the present disclosure includes a reusable, replaceable mask strap that may be used to securing the protective device to the patient. In an embodiment, the mask strap is also used to support a mask for administering gases (e.g., oxygen, nitrous oxide, or various inhalation anesthetics) to the patient before, during, or after a surgical or other medical procedure.

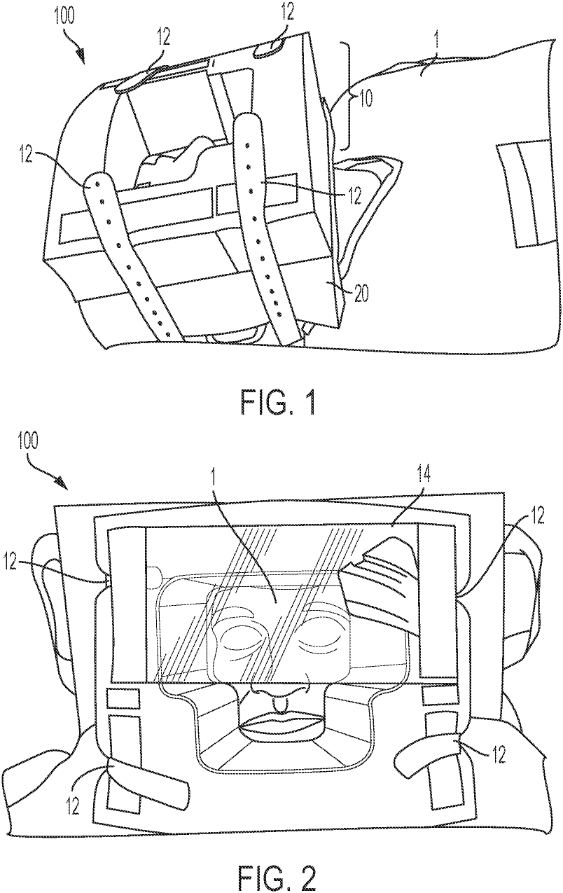

Referring to FIG. 1, a protective device 100 for protecting a face and/or head of an individual or patient 1 receiving medical treatment in a supine position is shown. The device 100 comprises a body 10 placed on the surface of the patient’s face and a strap 12 that is positioned behind the patient’s head and wraps partially upwardly around the sides thereof. FIG. 2 shows a top view of the device 100 on the patient 1 in the supine position with an eye protection shield 14 coupled to the body 10.

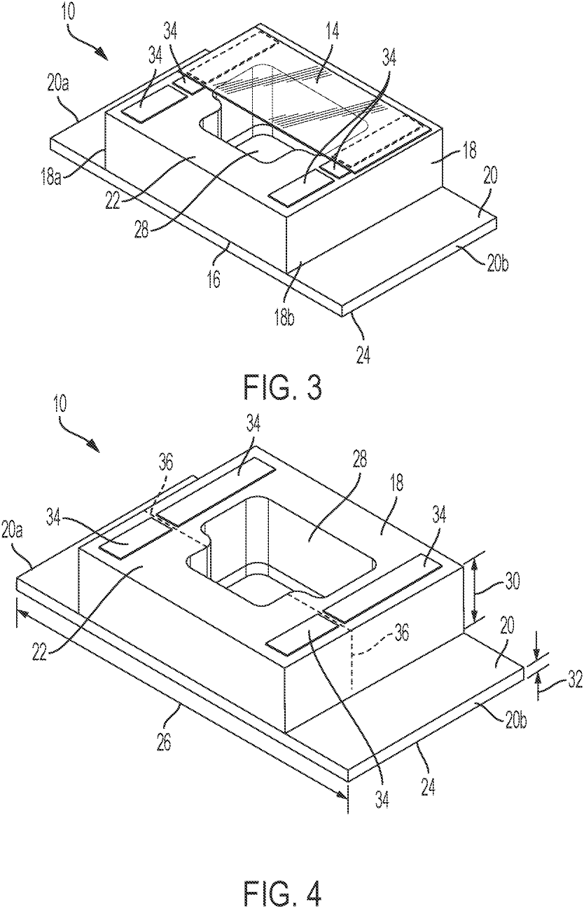

Referring to FIG. 3, a perspective view of the body 10 is shown in a relaxed state and not affixed to the patient 1. In one embodiment, the body 10 is comprised of two flat (e.g., planar) foam layers that are bonded together along a seam 16. In particular, the body 10 comprises a top portion 18 extending between a first top side edge 18a and a second top side edge 18b opposing the first side edge 18a and a base portion 20 coupled thereto along the seam 16. The base portion 20 includes opposing first and second bottom side edges 20a, 20b extending outwardly of the opposing first and second top side edges 18a, 18b. Opposite the seam 16, the top portion 18 defines an exterior surface 22 and the base portion 20 defines an interior surface 24. When affixed to a patient 1 (as shown in FIGS. 1 and 2), the exterior surface 22 faces away from the patient 1, while the interior surface 24 faces toward the patient 1 and is in proximate contact with the patient’s face and/or head.

Referring to FIG. 4, the body 10 is shown in perspective view and the eye protection shield 14 of FIG. 3 is removed for clarity. A width 26 dimension of the base portion 20 is selected to extend wider than the distance between the first side edge 18a and the second side edge 18b and thus the base portion 20 extends past the side edges 18a, 18b of the top portion 18. The width 26 is selected based on the circumference of the patient’s head and allows the base portion 20 to be deflected and bent around the sides of the patient’s head without extending beneath the head. In the embodiment illustrated in FIGS. 3 and 4, an aperture 28 extends between the exterior surface 22 and the interior surface 24 to provide exposure and access to the individual’s eyes, nose, and/or mouth.

The base portion 20 may be made from a soft foam material that helps to reduce abrasiveness or roughness against the patient’s face, while also providing effective comfort and pressure management as loads applied to the base portion 20 are distributed. The bending stiffness (e.g., rigidity) of the base portion 20 material is also selected such that the portions of the base portion 20 away from the seam 16 are flexible and capable of wrapping partially around the patient’s head. In an example, the base portion 20 foam may be a pneumatic foam, a “non-memory” foam, a viscoelastic “memory” foam, a slow recovery foam, a polyurethane foam, a fire retardant polyurethane foam, or a plastic or polymeric material. In some embodiments, the material of the base portion 20 provides at least some indentation load deflection, correlating to a minimum amount of weight needed to depress the material. In one embodiment, the base portion 20 has an indentation load deflection of between about 15 and about 25. In some embodiments, the material includes a viscoelastic gel having a viscosity that changes depending on the environment. For example, a material may become more stiff in cooler environments such as an operating room, requiring direct warmth from the patient to decrease the viscosity and become pliable. Additionally, the interior surface 24 of the base portion 20 interfacing with the patient 1 may have a high stiction factor to reduce the risk of slipping.

As an example, Table 1 below provides example foam properties that may be used for the foam of the base portion 20.

TABLE 1

| | Minimum | Max |
|---|---|---|
| Density, lb./ ft^3 (kg/m^3) | 2.35 (37.6) | 2.65 (42.4) |
| 25% Indentation Force Deflection (IFD) (for 15x15x4 in sample), lb. (N) | 12 (53.4) | 17 (75.6) |
| Tensile, psi (kpa) | 6 (41.4) | |
| Elongation, % | 120 | |
| Tear, ppi (N/mm) | 0.5 (0.088) | |
| Support Factor | 1.6 | |
| Hysteresis, % | | 25 |
| Ball rebound, % | 20 | |
| Ball rebound, % (memory foam) | <10 | |

The top portion 18 may also be made of a foam material that provides further protection and pressure management to the patient’s face by distributing localized forces applied to the exterior surface 22. In an example, the foam of the top portion 18 may be a pneumatic foam, a “non-memory” foam, a viscoelastic “memory” foam, a polyurethane foam, a fire retardant polyurethane foam, or a plastic or polymeric material. In one embodiment, the top portion 18 has an indentation load deformation of between about 40 and about 45. For example, the material of the top portion 18 may be a 1.2 lb polyurethane. In some embodiments, the foam of the top portion 18 may be selected to have a higher density and/or a higher stiffness than the foam of the base portion 20, and is therefore more resistant to bending, pressure, or other deformation than the base portion 20. The foam of the top portion 18 may also have a thickness 30 that is greater than a thickness 32 of the foam of the base portion 20. In an embodiment, the thickness 30 of the top portion 18 is approximately twice the thickness 32 of the base portion 20. In another embodiment, the thickness 30 of the top portion 18 is more than twice the thickness 32 of the base portion 20. By using thicker, denser, and/or higher stiffness foam for the top portion 18, relative to the base portion 20, the bending stiffness of the top portion 18 will be higher than the bending stiffness of the base portion 20. As best shown in FIG. 1, the stiffness of the top portion 18 allows the device 100 to bend and conform to the curvature of the surface of the patient’s face, while the stiffness of the base portion 20 allows the device 100 to bend into conforming contact with the sides of the patient’s head. By placing of the base portion 20 along the sides of the head, rather than also behind the head, the device 100 promotes rotational stability so that the patient’s face maintains a generally upwards orientation.

Alternatively, the top portion 18 and the base portion 20 may also be formed from a single type of foam in some embodiments. For example, the base portion 20 may include cavities or openings to reduce stiffness and allow the side portions to bend easily around the patient’s head. Still further, some embodiments may omit the seam 16 and the top portion 18 may be monolithically formed with the base portion 20 from a single piece of foam.

Referring still to FIG. 4, the top portion 18 further comprises a plurality of connection positions 34 located along the exterior surface 22. In the example of FIG. 4, the plurality of connection positions 34 comprises four segments of hook fabric (such as the fabric sold under the trademark Velcro®) adhesively bonded to the exterior surface 22 along a perimeter of the exterior surface 22 proximate to the side edges 18a, 18b. As detailed further herein, the plurality of connection positions 34 serve as anchoring positions for the strap 12 (shown in FIGS. 1 and 2) and for the eye protection shield 14 (shown in FIG. 2). In other embodiments, the plurality of connection positions 34 may include rings through which ends of the strap 12 extend, protrusions or hooks that engage with openings on the ends of the strap 12, or any other suitable mechanism for attaching the strap 12 to the top portion 18 of the body 10.

Additionally, the top portion 18 may further comprise one or more slits 36 that extend from the exterior surface 22 toward the base portion 20. The slits 36 may be of varied depth or may be a constant depth. In an embodiment, the slits 36 only extend partially through the thickness 30 of the top portion 18. The slits 36 are formed by parting (e.g., slicing, sawing, etc.) the top portion 18 such that minimal material is removed by the kerf of the parting tool and thus the opposing surfaces forming the slit 36 remain in close proximate contact or direct touching contact when the body 10 is in the relaxed state. In an example, the opposing surfaces forming the slits 36 are spaced apart 1/32 inches (0.79 mm) or less. Alternatively, the opposing surfaces forming the slits 36 are spaced apart 1/64 inches (0.40 mm) or less. Alternatively, the opposing surfaces forming the slits 36 are not spaced apart, and are thus in direct touching contact. During use, the slits 36 are configured to accept accessory items (e.g., tubing, electrically conductive wires, medical equipment, other accessories, etc.) and allow the positioning of the accessory items to be fully or partially below the exterior surface 22 of the top portion 18. In addition, the opposing surfaces forming the slits 36 may be configured to frictionally grip the accessory items placed within the slits 36.

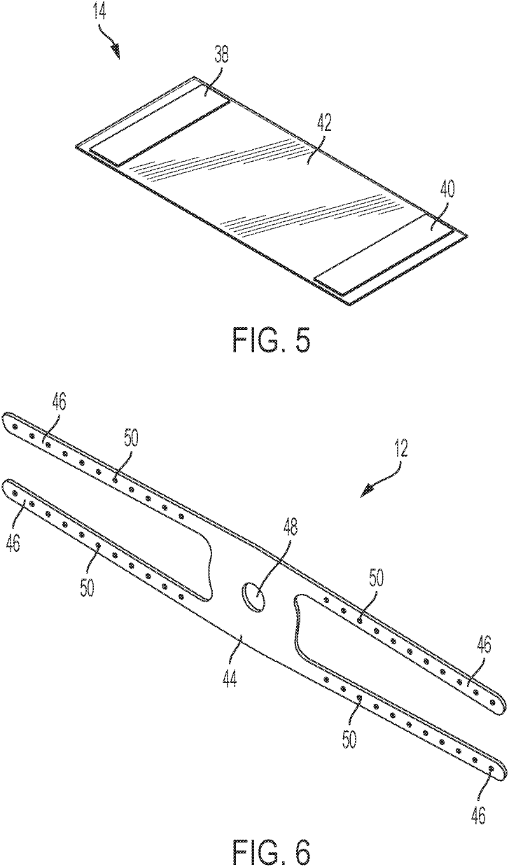

Referring to FIG. 5, the eye protection shield 14 is shown in a perspective view from below. A first pad 38 and a second pad 40 are positioned on opposing ends of an inner surface 42. During use of the device 100 (as best shown in FIGS. 2 and 3) the eye protection shield 14 may be detachably coupled along the inner surface 42 to the exterior surface 22 of the top portion 18 to at least partially obstruct or block the aperture 28. To secure the positon of the eye protection shield 14, the pads 38, 40 may couple to the connection positions 34 along the exterior surface 22 of the top portion 18. In an example, the pads 38, 40 are made from loop material that removably attaches with hook material on the connection positions 34. The eye protection shield 14 may be formed from transparent, translucent, or opaque material. In an example, the eye protection shield 14 is made from a clear, fog-free polyethylene terephthalate (“PET”) or similar plastic sheet material to allow monitoring of the patient’s eyes. In addition, by obstructing the aperture 28, the patient’s eyes are protected against items that may fall or protrude into the aperture 28. The material of the eye protection shield 14 may be flat or curved in a relaxed state (e.g., when not subjected to flexure in bending), and may be configured to conform to the curvature of the body 10 when coupled to the patient 1. As described further herein, the eye protection shield 14 is typically coupled to the body 10 after the body 10 is coupled to the patient’s face via the strap 12, and thus the pads 38, 40 of the eye protection shield 14 may at least partially cover a portion of the strap 12 that is coupled to the connection positions 34.

Referring to FIG. 6, the strap 12 is shown in a perspective view and comprises a base 44 coupled to four arms 46. The base 44 comprises a central hole 48 and each of the four arms 46 comprises a plurality of holes 50 distributed along their length. During use of the strap 12, the base 44 is placed behind the patient’s head. During placement, the central hole 48 may be used to locate a generally central position of the patient’s head. The arms 46 are then sequentially or concurrently attached to the connection positions 34 along the exterior surface 22 of the body 10 using any desirable connection order. Tension is applied to the strap 12 as the arms 46 are connected and as the base portion 20 of the body 10 is bent into conforming contact with the patient’s face. As best shown in FIG. 1, the arms 46 of the strap 12 also bend and hold the base portion 20 into conforming contact with the sides of the patient’s head.

The arms are connected to the side edges of the top portion in order to equalize the pressure applied to the patient’s face along the interior surface 24 of the body 10. The plurality of connection positions 34 in combination with the interior surface 24 having a high stiction factor also better anchors the body 10 onto the patient’s face so that the protection device 100 does not shift or slip during operation.

In an example, the strap 12 is formed from a continuous piece of loop fabric such as Veltex® fabric that secures easily to the hook material of the connection positions 34 of the body 10. Thus the arms 46 may be connected to the connection positions 34 along an infinite number of positions along the lengths of the arms 46 to adjust the tension on the strap 12. In other embodiments, the strap 12 is formed of a flexible material that includes hook and loop material near the ends of the arms 46 such that the arms 46 extend through rings of the connection positions 34 and are tightened using the hook and loop attachment means at each end. In another embodiment, the strap 12 includes openings on the arms 46 that engage with protrusions or hooks at the connection positions 34 of the body 10. In still further embodiments, the arms 46 of the strap 12 may include any other suitable mechanism for attaching the strap 12 to the top portion 18 of the body 10.

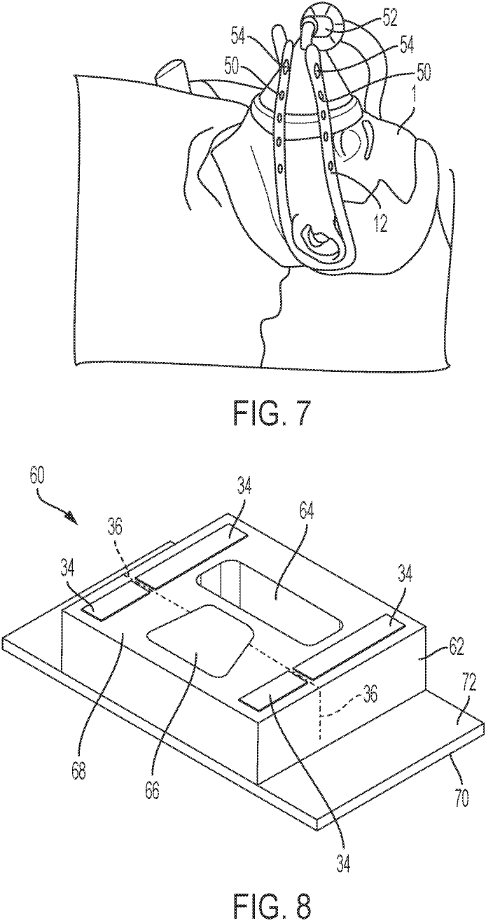

In addition, the strap 12 may also be used during a medical treatment before the body 10 is affixed to the patient 1 or after the body 10 is removed from the patient 1. In particular, as shown in FIG. 6, the strap 12 may be configured to also attach with an anesthesia face mask 52 so that the strap 12 is already in position behind the patient’s head before or after the body 10 is needed. The series of holes 50 may be secured to a plurality of retention clips 54 to secure the anesthesia face mask 52 in position as shown in FIG. 7. The anesthesia face mask 52 may then be used to administer gases (e.g., oxygen, nitrous oxide, or various inhalation anesthetics) to the patient 1. The anesthesia provider may then further anesthetize the patient 1 (e.g., via intravenous sedation), remove the anesthesia face mask 52, intubate the patient 1, and may then use the strap 12 to secure the body 10 to the patient 1.

Referring to FIG. 8, a body 60 according to another embodiment is shown that may be used in place of the body 10 previously described. The body 60 is generally similar to the body 10 and thus similar reference numerals and/or names are used to identify similar features and the explanation of such features will not be repeated in the interest of brevity except as needed for clarity. Instead, the description will focus primarily on aspects of the body 60 that are different from the body 10. In particular, a top portion 62 comprises a first aperture 64 and a second aperture 66. The first aperture 64 extends between an exterior surface 68 of the top portion 62 and an interior surface 70 of a base portion 72 to provide exposure for the patient’s eyes. In addition, the second aperture 66 extends between the exterior surface 68 and the interior surface 70 to provide exposure for the patient’s nose and mouth. The strap 12 may then be used as previously described to affix the body 60 to the patient 1, and the eye protection shield 14 may be attached to partially or fully obstruct the first aperture 64. Because the access to the nose and mouth of the patient 1 is provided by the second aperture 66, the first aperture 64 may be fully obstructed by the eye protection shield 14 and thus may provide potentially superior eye protection relative to the body 10 previously described that only partially obstructed the aperture 28.

The device 10, 60 and method of using the device 10, 60 according to the present disclosure provides protection for the patient 1 undergoing surgery in the supine or reclined positions by allowing forces to the patient’s head or face to be distributed. A flexible foam construction also provides a relatively light weight device 100 that promotes the prevention of injury to the patient 1 relative to stiffer and/or heavier protective devices. In addition, the placement of the base portions 20, 72 along the sides of the head, rather than also behind the head, also promotes a rotationally stable construct that maintains the position of the patient’s head. By maintaining the patient’s head in a stable position, the device 100 and method disclosed also further reduces the risks of decreased blood flow and possible nerve damage. Still further, by using the strap 12 that can connect to both the anesthesia face mask 52 and to the bodies 10, 60, the strap 12 may stay in place as the patient’s head remains in a stationary and stable position, and thus still further reduce the risks of decreased blood flow and possible nerve damage. The device 10, 60 and methods of using the device 10, 60 therefore, provide increased protection to the patient 1 over conventional practices.

Without further elaboration, it is believed that one skilled in the art can use the preceding description to utilize the claimed inventions to their fullest extent. The examples and embodiments disclosed herein are to be construed as merely illustrative and not a limitation of the scope of the present disclosure in any way. It will be apparent to those having skill in the art that changes may be made to the details of the above-described embodiments without departing from the underlying principles discussed. In other words, various modifications and improvements of the embodiments specifically disclosed in the description above are within the scope of the appended claims. For example, any suitable combination of features of the various embodiments described is contemplated.

What is claimed is:

1. A device for protecting a face and/or head of an individual receiving medical treatment in a supine or reclined position, the device comprising:
a body including a top portion with an exterior surface and a base portion with an interior surface, wherein the top portion includes opposing top side edges, wherein the base portion includes opposing bottom side edges extending outwardly from the top side edges, wherein the base portion is flexible and is configured to wrap partially around the individual’s head, wherein the body includes an aperture extending between the exterior surface and the interior surface to provide access to at least one of the individual’s eyes, nose, or mouth; and
a strap spanning the side edges of the top portion of the body and configured to wrap behind the base portion and the individual’s head to secure the interior surface of the base portion into proximate contact with the face and/or head, and wherein the strap is configured to detachably couple to the exterior surface of the top portion along a plurality of connection positions along the side edges.

2. The device of claim 1, further comprising an eye protection shield configured to detachably couple to the exterior surface of the top portion and to at least partially obstruct the aperture.

3. The device of claim 1, wherein the plurality of connection positions comprises four connection points on the exterior surface of the top portion, and wherein the strap is formed from a continuous piece of material.

4. The device of claim 1, wherein the plurality of connection positions comprises four segments of hook fabric on the exterior surface of the top portion, and wherein the strap is formed from a continuous piece of loop fabric and is configured to equalize pressure between the plurality of connection positions.

5. The device of claim 1, wherein the top portion is monolithically formed with the base portion.

6. The device of claim 1, wherein the top portion is thicker than the base portion.

7. The device of claim 1, wherein the exterior surface further comprises a slit extending partially through a thickness of the top portion, wherein opposing surfaces forming the slit are configured to frictionally grip tubing or electrically conductive wires placed within the slit.

8. The device of claim 1, wherein the top portion and the base portion are adhesively coupled.

9. The device of claim 8, wherein the top portion is formed from a first foam that is stiffer than a second foam forming the base portion.

10. A device for protecting a face and/or head of an individual receiving medical treatment in a supine or reclined position, the device comprising:
a body comprising a top portion with an exterior surface and a base portion with an interior surface, wherein the top portion includes opposing side edges, wherein the base portion includes opposing bottom side edges extending outwardly from the top side edges, wherein the base portion is flexible and is configured to wrap partially around the individual’s head, wherein the top portion is not configured to wrap partially around the individual’s head;
wherein the body includes a first aperture extending between the exterior surface and the interior surface to provide access to the individual’s eyes, and a second aperture extending between the exterior surface and the interior surface to provide access to the individual’s nose and mouth; and
a strap spanning the side edges of the top portion of the body and configured to wrap behind the individual’s head to secure the interior surface of the base portion into proximate contact with the face and/or head, and wherein the strap is configured to detachably couple to the exterior surface of the top portion along a plurality of connection positions.

11. The device of claim 10, wherein the plurality of connection positions comprises four segments of hook fabric on the exterior surface of the top portion, and wherein the strap is formed from a continuous piece of loop fabric.

12. The device of claim 10, wherein the top portion is monolithically formed with the base portion.

13. The device of claim 10, wherein the exterior surface further comprises a slit extending partially through a thickness of the top portion, wherein opposing surfaces forming the slit are configured to frictionally grip tubing or electrically conductive wires placed within the slit.

14. The device of claim 10, wherein the top portion and the base portion are adhesively coupled.

15. The device of claim 14, wherein the top portion is formed from a first foam that is stiffer than a second foam forming the base portion.

16. The device of claim 10, further comprising an eye protection shield configured to detachably couple to the exterior surface of the top portion and fully obstruct the first aperture.

17. The device of claim 16, wherein the eye protection shield is formed from a clear and fog-free material.

18. A method of protecting a face and/or head of an individual receiving medical treatment in a supine or reclined position, the method comprising:
providing a device with a body comprising a top portion and a base portion, wherein the top portion includes opposing side edges, wherein the base portion includes opposing bottom side edges extending outwardly from the top side edges;
providing a strap behind the individual’s head, wherein opposing ends of the strap extend beyond the sides of the individual’s head;
placing an interior surface of the base portion into proximate contact with the face and/or head of the individual;
positioning the body so that an aperture provides access at least one of the individual’s eyes, nose, or mouth to an exterior surface;
wrapping at least a portion of the base portion partially around the individual’s head, wherein the top portion is not configured to wrap partially around the individual’s head;
attaching the ends of the strap to the side edges of the top portion of the device and coupling the strap to the exterior surface of the top portion along a plurality of connection positions to secure the body to the individual’s face and/or head.

19. The method of claim 18, further comprising coupling an eye protection shield to the exterior surface of the top portion to at least partially obstruct the aperture.

20. The method of claim 18, further comprising:
pressing tubing or electrically conductive wires into a slit formed in the exterior surface of the top portion, wherein the slit extends partially through a thickness of the top portion; and
holding the tubing or electrically conductive wires in a fixed position with friction of opposing surfaces forming the slit.
